(19) United States
(12) Patent Application Publication
(10) Pub. No.: US 2025/0209626 A1
(43) Pub. Date: Jun. 26, 2025

(54) METHODS AND SYSTEMS FOR AUTOMATIC CT IMAGE QUALITY ASSESSMENT

(71) Applicant: GE Precision Healthcare LLC, Waukesha, WI (US)

(72) Inventors: Mathieu Bedez, Strasbourg (FR); Philippe Gerner, Strasbourg (FR); Jean Baptiste Wahl, Strasbourg (FR)

(21) Appl. No.: 19/078,180

(22) Filed: Mar. 12, 2025

Related U.S. Application Data
(63) Continuation-in-part of application No. 18/485,134, filed on Oct. 11, 2023.
(60) Provisional application No. 63/384,383, filed on Nov. 18, 2022.

(51) Int. Cl. G06T 7/00; G06T 7/12; G16H 30/20

(52) U.S. Cl. CPC G06T 7/0014; G06T 7/12; G16H 30/20; G06T 2207/10081; G06T 2207/20081; G06T 2207/30168

(57) ABSTRACT

Methods and systems are provided for automatically evaluating an image quality of medical images, and collecting user feedback with respect to the image quality via a graphical user interface (GUI). The tool provides an assessment of noise, contrast, and other characteristics of slices of an image associated with organs and/or anatomical structures of the image based on various low-level metrics, and calculates an overall image quality score of the image by aggregating the image scores of the organs and/or anatomical structures. The score may be estimated by a machine learning (ML) model based on the low-level metrics. The user can provide feedback with respect to the image quality score, which may be used to increase an accuracy and/or performance of the ML model. In this way, feedback may be efficiently collected and used to refine the ML model, leading to more accurate scan protocols and higher quality CT images.

### CROSS-REFERENCE TO RELATED APPLICATION

The present application is a continuation-in-part of U.S. patent application Ser. No. 18/485,134, entitled “METHODS AND SYSTEMS FOR AUTOMATIC CT IMAGE QUALITY ASSESSMENT,” and filed Oct. 11, 2023. U.S. patent application Ser. No. 18/485,134 claims priority to U.S. Provisional Application No. 63/384,383, entitled “METHODS AND SYSTEMS FOR AUTOMATIC CT IMAGE QUALITY ASSESSMENT,” and filed on Nov. 18, 2022. The entire contents of each of the above-listed applications are hereby incorporated by reference for all purposes.

### TECHNICAL FIELD

Embodiments of the subject matter disclosed herein relate to medical imaging, and in particular, assessing a quality of computed tomography images.

### BACKGROUND

In a computed tomography (CT) imaging system, an electron beam generated by a cathode is directed towards a target within an X-ray tube. A fan-shaped or cone-shaped beam of X-rays produced by electrons colliding with the target is directed towards an object, such as a patient. After being attenuated by the object, the X-rays impinge upon an array of radiation detectors, generating an image.

The high quality of the image is essential for achieving accurate and reliable patient diagnostics. The quality of the image may be assessed in terms of spatial resolution, contrast, noise, a presence and/or number of artifacts generated, and/or other characteristics. The quality of the image may depend on various acquisition parameters of the CT imaging system configured by a user of the CT imaging system, such as radiation dosage (e.g., an amount of current applied to the cathode), focal spot size and/or shape; sampling frequency; a selected field of view; slice thickness, a selected reconstruction algorithm; and others. As a result, image quality may not be consistent across users and/or scans performed, where some parameter configurations may generate higher quality images than other parameter configurations. As a result, to generate an image of a desired quality, various scans may be performed on a patient where parameters are adjusted in a trial and error fashion, resulting in an increased consumption of imaging system resources and higher radiation exposures for patients. Feedback regarding how the CT imaging system could be differently configured to increase the quality of reconstructed images may not be available, and learning how to configure the settings to consistently generate high quality images may entail years of user experience via a trial and error process.

As relevant technologies evolve, users increasingly request and expect higher levels of automation from radiology applications, where artificial intelligence (AI) techniques are used to automate aspects of a CT system and aid users in obtaining desired results.

### SUMMARY

The current disclosure at least partially addresses one or more of the above identified issues by a method for an image quality assessment system, the method comprising receiving medical image series data from a user of the image quality assessment system; generating an image quality score for a medical image included in the image series data, the image quality score generated using a trained machine learning (ML) model; displaying the selected medical image and the image quality score in a graphical user interface (GUI) on a display device of the image quality assessment system; receiving an adjusted image quality score of the medical image from the user via the GUI; and using the adjusted image quality score to retrain the ML model; wherein the image quality score is based on aggregated image quality scores calculated for one or more organs and/or anatomical structures in the medical image.

The current disclosure further includes a medical imaging system comprising a display device, the medical imaging system being configured to display on the display device a menu listing a plurality of slices of a 3-D medical image viewable on the display device; and additionally being configured to display on the display device an image quality graphical user interface (GUI) that can be reached directly from the menu; wherein the image quality GUI displays, for a set of slices of the plurality of slices, an image quality score generated by an image quality score generator, and a limited list of results of low-level image quality metrics applied to the set of slices to generate the image quality score, the low-level image quality metrics applied to image data of one or more organs of each slice and not to image data outside the one or more organs using an organ mask, each result in the limited list being selectable to launch a display panel with additional information relating to the low-level image quality metrics applied to the set of slices and enable at least the selected result to be seen within the display panel, and wherein the image quality GUI is displayed while the image quality score generator is in an unlaunched state.

The current disclosure further includes an image quality assessment system, comprising: a processor; and a memory storing instructions that when executed, cause the processor to: receive a Digital Imaging and Communications in Medicine (DICOM) file including medical image series data acquired from a medical imaging system from a user of the image quality assessment system; extract a name of a protocol used to acquire the medical image series data from metadata of the DICOM file; perform a segmentation of a plurality of organs and/or anatomical structures included in the protocol to determine boundaries of the organs and/or anatomical structures; calculate an image quality score for each organ and/or anatomical structure of the organs and/or anatomical structures in each slice of each image of the medical image series data using a machine learning (ML) model, the ML model taking as input a plurality of low-level image quality metrics applied to each organ and/or anatomical structure of each slice; aggregate image quality scores of each organ and/or anatomical structure of the organs and/or anatomical structures to generate an overall image quality score of the medical image series data; display a medical image of the medical image series data to a user of the image quality assessment system via a graphical user interface (GUI) of the image quality assessment system; display the overall image quality score of the medical image in the GUI; receive an adjusted image quality score of the medical from the user via the GUI; and retrain the ML model using training data including the adjusted image quality score.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a schematic block diagram of an image quality assessment system;

FIG. 2 is a schematic block diagram indicating an exemplary strategy for generating an image quality score of a medical image;

FIG. 3 is a schematic block diagram showing an overview of steps of a procedure for generating the image quality score;

FIG. 4 is a flowchart illustrating an exemplary method for generating and displaying the image quality score;

FIG. 5 is a flowchart illustrating an exemplary method for ingesting and processing one or more Digital Imaging and Communications in Medicine (DICOM) files;

FIG. 6 is a flowchart illustrating an exemplary method for displaying image quality scores on a display device and receiving user feedback;

FIG. 7 is an exemplary image quality GUI of the image quality assessment system;

FIG. 8 shows an exemplary display panel that may be used to navigate to desired images included in one or more studies generated using a medical imaging system;

FIG. 9 is an example of a display panel indicating low-level image quality metrics for a plurality of slices of an image, displayed in the GUI of the image quality assessment system;

FIG. 10 is an example of a display panel showing a slice of a medical image displayed in the GUI of the image quality assessment system;

FIG. 11 is a flowchart illustrating an exemplary method for detecting and segmenting organs and/or anatomical structures in a medical image; and

FIG. 12 shows an example of a display panel that indicates a noise score for a plurality of organs of a medical image series.

### DETAILED DESCRIPTION

The methods and systems described herein relate to automatically generating an assessment of a quality of a medical image acquired via a medical imaging system, such as a computed tomography (CT) image acquired via a CT system. While descriptions of the systems and methods herein may refer to CT images and systems, it should be appreciated that the systems and methods may also apply to other types of medical images. The assessment may be used to optimize acquisition parameters of the medical imaging system for various imaging tasks and scenarios. For example, image quality assessments may be used to train one or more machine learning (ML) models to predict a set of acquisition parameters for an imaging task that maximizes the quality of images reconstructed using the set of acquisition parameters.

A quality of a CT image may be degraded due to distortions during image acquisition and processing. Examples of distortions include noise, blurring, and/or a presence of artifacts such as ring artifacts, compression artifacts (e.g., graininess of an image), and/or streaking. The quality of a distorted or degraded CT image may be evaluated using various metrics, which may estimate noise and/or contrast levels, spatial resolution, or other aspects of the CT image.

For example, the quality of a CT image can be evaluated with respect to noise. Noise in a CT image is based on photon counting statistics, where a level of noise in the image is inversely related to a number of photons counted at a detector of the CT image (e.g., noise in the image decreases as a number of counted photons increases). Thus, the image noise is dependent on a radiation dosage, where increasing the dosage reduces noise. Noise in a CT image may be measured as a total amount of noise, or a signal-to-noise ratio (SNR). Other factors that affect the level of noise in a CT image are slice thickness, where thicker slices increase SNR; patient size, where larger patients reduce SNR; and a reconstruction algorithm selected to reconstruct the image, where uniform regions of the image may have lower noise than highly structured regions. Noise has various measurable aspects, including magnitude (e.g., the denominator of the SNR), texture (e.g., a quality of the noise, which may be measured by computing a noise power spectrum (NPS)), and uniformity (e.g., a measurement of variations in magnitude or texture across the image).

The quality of a CT image can also be evaluated with respect to contrast. Contrast is the difference between values in different parts of the image, which may allow for distinguishing between different types of tissues. Contrast is typically expressed in terms of a contrast-to-noise ratio (CNR), which is the ratio of the contrast between a signal in a given region and a background. The CNR can be calculated based on measurements within regions of interest (ROI) of the CT image. As the CNR increases, the ROI may be more easily visualized with respect to the background.

The quality of a CT image can also be evaluated with respect to spatial resolution. Spatial resolution refers to the ability to distinguish between objects of different densities in the image. A high spatial resolution may be relied on for discriminating between structures located within a close proximity to each other. Factors that impact spatial resolution include detector size, slice thickness, edge enhancement vs. soft tissue kernels, pitch, field of view (FOV), pixel size, and focal spot size, among others.

Various other metrics may be used to assess image quality. For many applications, a valuable quality metric correlates well with the subjective perception of quality by a human observer. One type of metrics, referred to as full-reference metrics, include comparing a first version of an image including distortions with a second, reference version of the image not including distortions. These metrics may include, for example, SNR, various structural similarity (SSIM) metrics, NPS, e.g., an intensity of noise as a function of spatial frequency (noise texture); visual information fidelity (VIFp), feature similarity index measurement (FSIM), spectral residual-based similarity (SR-SIM), gradient magnitude similarity deviation (GMSD), visual saliency-induced index (VSI), and deep image structure and texture similarity (DISTS), among others.

If no reference versions of the image are available, various non-reference metrics and/or models may be used to estimate a quality of an image based on expected image statistics. Examples of non-reference models include a natural image quality evaluator (NIQE) model, which is based on measuring deviations from statistical regularities in natural images; a perception-based image quality evaluator (PIQE) model, which is based on mean subtraction contrast normalization; and a Blind/Referenceless Image Spatial Quality Evaluator (BRISQUE) model, which is a scene statistic model based on locally normalized luminance coefficients of an image.

Objective image quality assessment methods may be based on various computed low-level quality metrics, also referred to herein as low-level metrics, including Noise Power Spectrum (NPS), SNR, CNR, SSIM, uniformity metrics, and others. SNR and peak SNR (PSNR) are based on capturing signal visibility drawn in noise on a noise map, where a higher (P)SNR is an indicator of higher image quality. SSIM is computed by comparing an image to a denoised (reference) image. Alternatively, a histogram flatness measure (HFM) and/or a histogram spread (HS) capture variations in contrast in an image, where a low HFM/HS value indicates low contrast in the image, and a high HFM/HS value indicates high contrast in the image.

To generate high quality CT images, various parameters of a CT system may be initially set at the beginning of a scan, where settings of the various parameters are established by a scan protocol of the scan. The established parameter settings may be established in the scan protocol by one or more human experts, based on experience, research, and/or predictive models (statistical models, AI/ML models, etc.), or the established parameter settings may be generated by the predictive models without the involvement of human experts. The predictive models may be trained using training data including a plurality of acquisition parameter settings and high quality CT images generated using the acquisition parameter settings as ground truth data. However, as collecting the training data may be cumbersome and time consuming, available training data may be insufficient.

As a result, the parameter settings established in the scan protocol may not generate images of high quality, and the parameters may be adjusted by an operator of the CT system, before or during the scan. For example, a scanned subject may be larger than a typical scanned subject, and the operator may adjust one or more parameters based on the larger size of the scanned subject. If an amount of noise observed in a reconstructed image is higher than desired, a radiation dosage applied by the CT system may be increased. If a spatial resolution of the image is lower than desired, a size of a focal spot of the CT system may be decreased, and/or an FOV of the CT system may be decreased, or a different parameter may be adjusted. Thus, generating high quality CT images may depend on first assessing the quality of a reconstructed image, and based on the assessment, setting specific parameters of the CT system to achieve a desired quality.

The predictive models used by the one or more human experts to generate recommended parameter settings for scan protocols and/or recommendations for adjusting the parameter settings of the scan protocols may be periodically adjusted and/or retrained, which may result in improved recommended settings that result in images of increased quality. However, generating image quality data suitable for retraining the models may rely on efficiently collecting image quality assessment feedback from a plurality of operators of the CT system. For the image quality data to be suitable, a desired specificity of the feedback may be high. For example, an image quality assessment for an ROI may be different from an image quality assessment of a slice of the image including the ROI, which may be different from an image quality assessment of the entire image. Additionally or alternatively, an image quality of a first ROI of an image may be different from an image quality of a second ROI of the image.

To efficiently collect data with this degree of specificity, the inventors herein have developed an assessment tool for automatically evaluating image quality of medical images, and collecting operator feedback with respect to the image quality via a proposed image quality graphical user interface (GUI). The tool assesses noise, texture, contrast, resolution, and other characteristics of slices of an image, anatomical regions of the image, and/or ROIs of the image based on various low-level metrics, and calculates an overall image quality score of the image by aggregating the low-level metrics. Specifically, the score may be estimated by a machine learning (ML) model based on the low-level metrics. The user can provide feedback with respect to the image quality score, which may be used to increase an accuracy and/or performance of the ML model. In this way, feedback may be efficiently collected and used to refine the ML model, which may allow for a more accurate image quality score.

The systems and methods described herein provide specific technical improvements to computer-based medical image quality assessment systems. In particular, the disclosed techniques improve the efficiency and reduce the computational burden of generating and displaying organ-specific image quality metrics by implementing a two-phase technical approach: (1) pre-calculating quality scores and metrics using machine learning models and organ segmentation masks during an initial processing phase, and (2) enabling rapid retrieval and display of the pre-calculated metrics while maintaining the ML model in an unlaunched state during user interaction. This technical architecture avoids the need to repeatedly execute computationally intensive ML model calculations each time a user selects different organs or image regions to analyze. The organ segmentation masks enable more accurate and focused quality metric calculations by precisely isolating relevant image data within organ boundaries while excluding irrelevant surrounding data. The interactive GUI elements are specifically configured to leverage the pre-calculated metrics architecture by retrieving and aggregating the stored organ-specific scores on-demand without relaunching the ML scoring engine. This provides a technical solution that enables responsive real-time updates to quality visualizations as users navigate between different organs and regions, while minimizing system computational load. The automated collection and storage of user feedback adjustments to the ML-generated scores creates a technical feedback loop that continuously improves the accuracy of the underlying ML models through targeted retraining. Additionally, the system's ability to map between different organ naming conventions (e.g., RadLex to segmentation software) and automatically extract protocol information from DICOM metadata enables seamless integration across different medical imaging platforms and systems. These technical features work together to create an efficient and scalable solution for assessing and improving medical image quality that reduces computational overhead, enables rapid user interaction, and drives continuous ML model improvement through automated feedback collection.

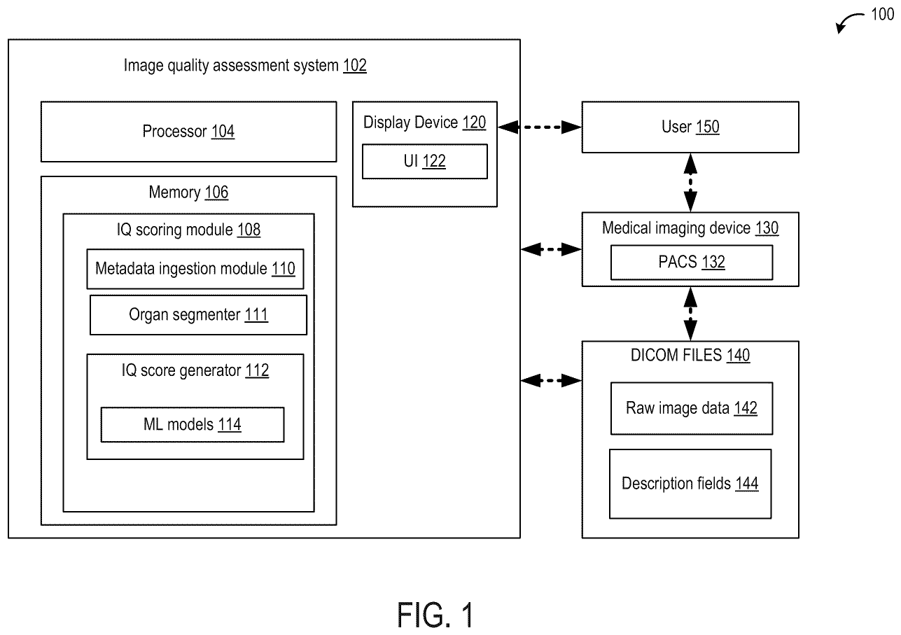

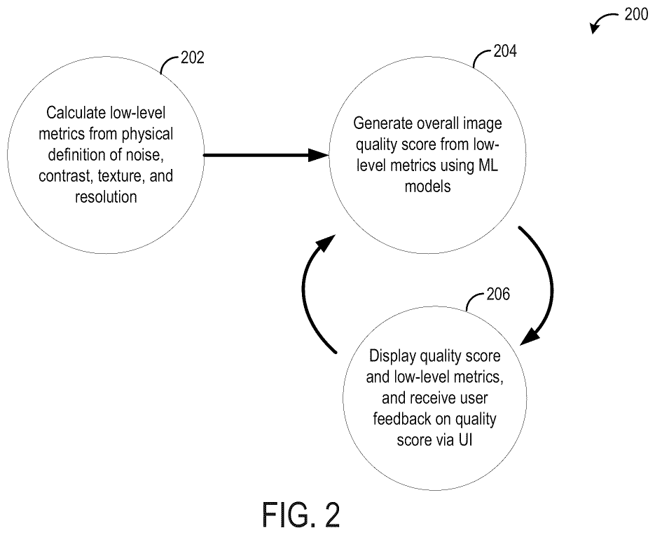

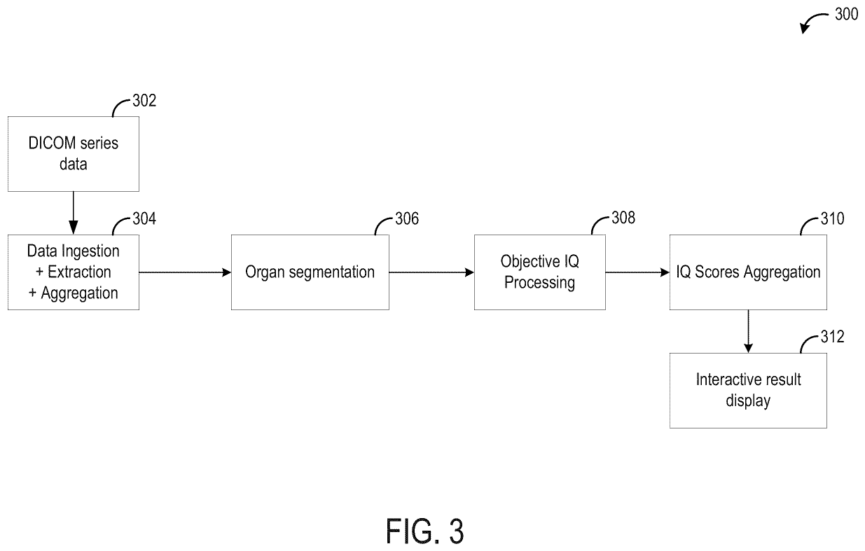

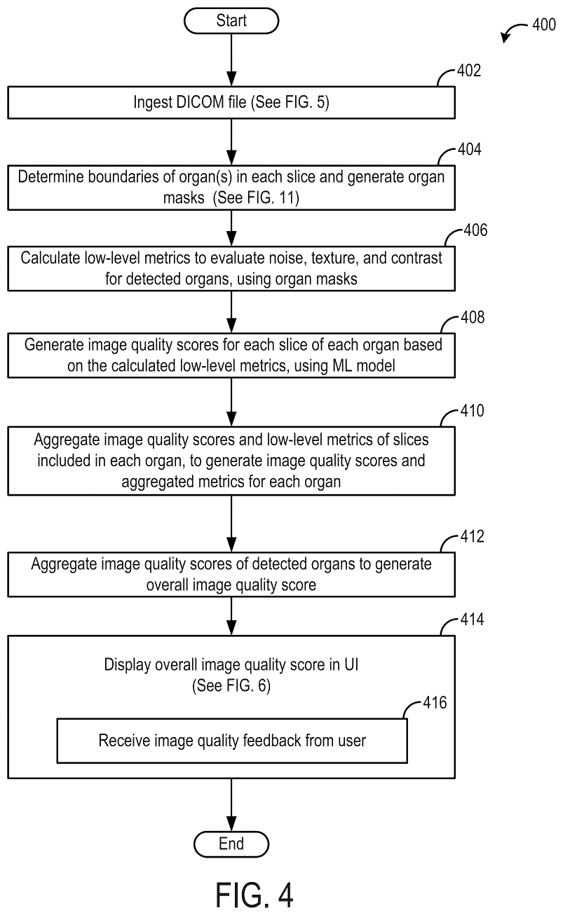

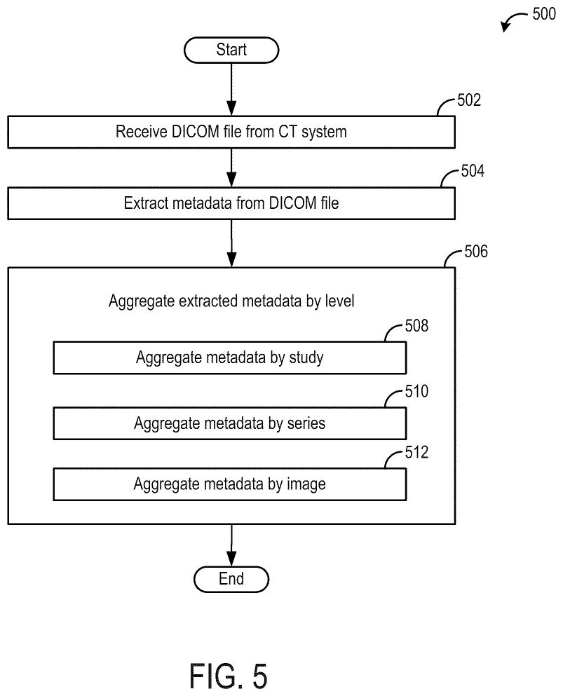

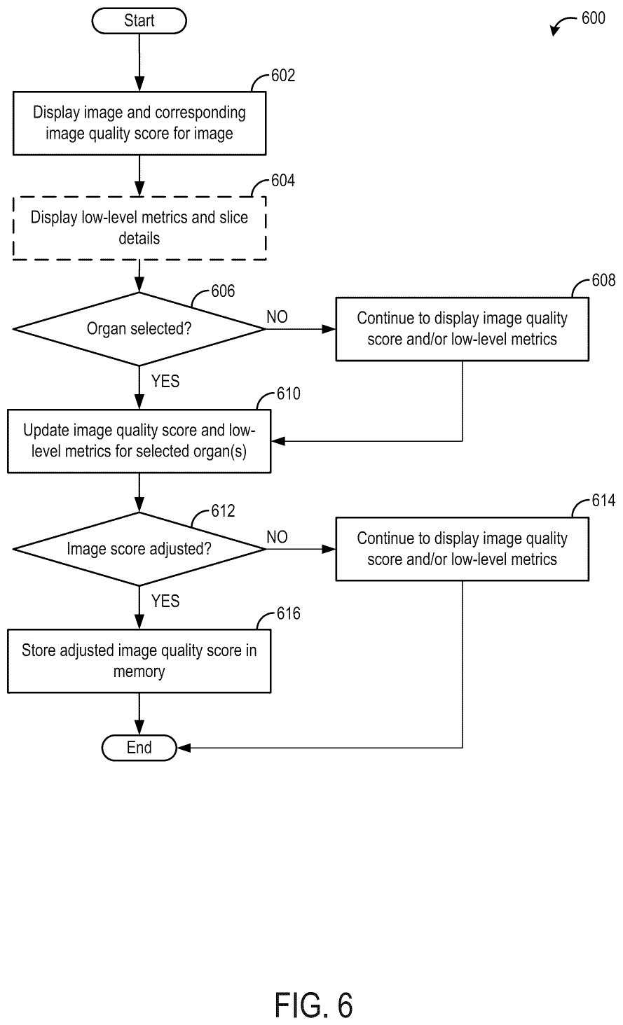

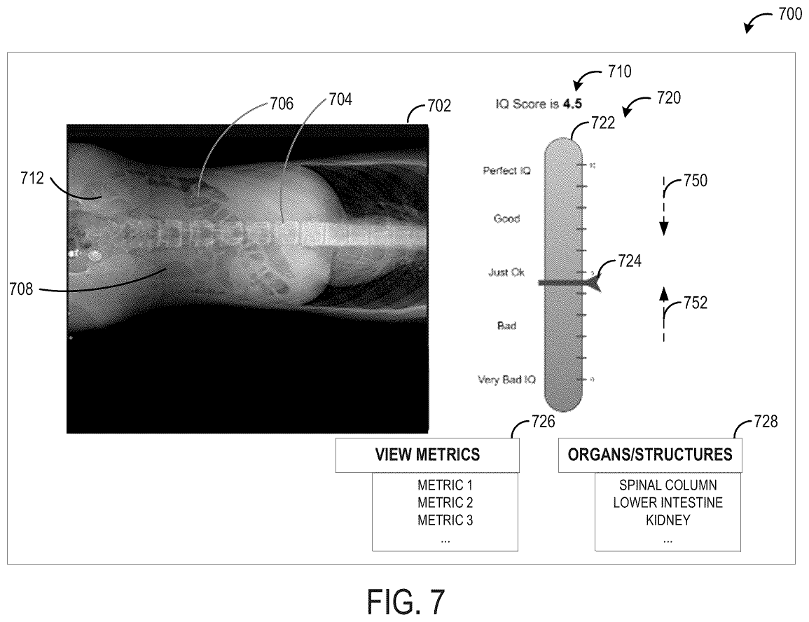

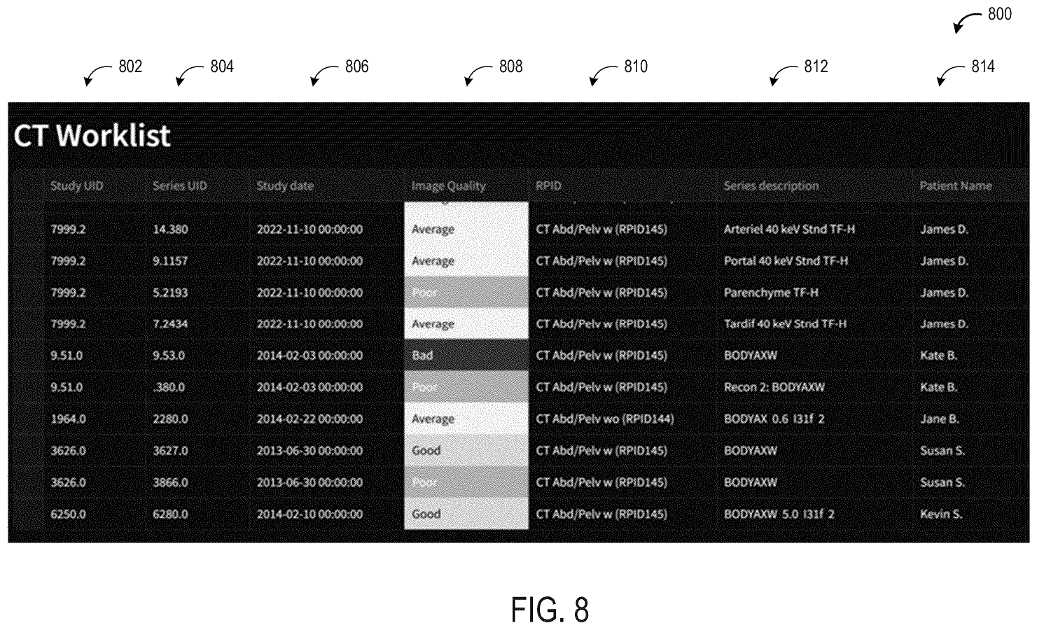

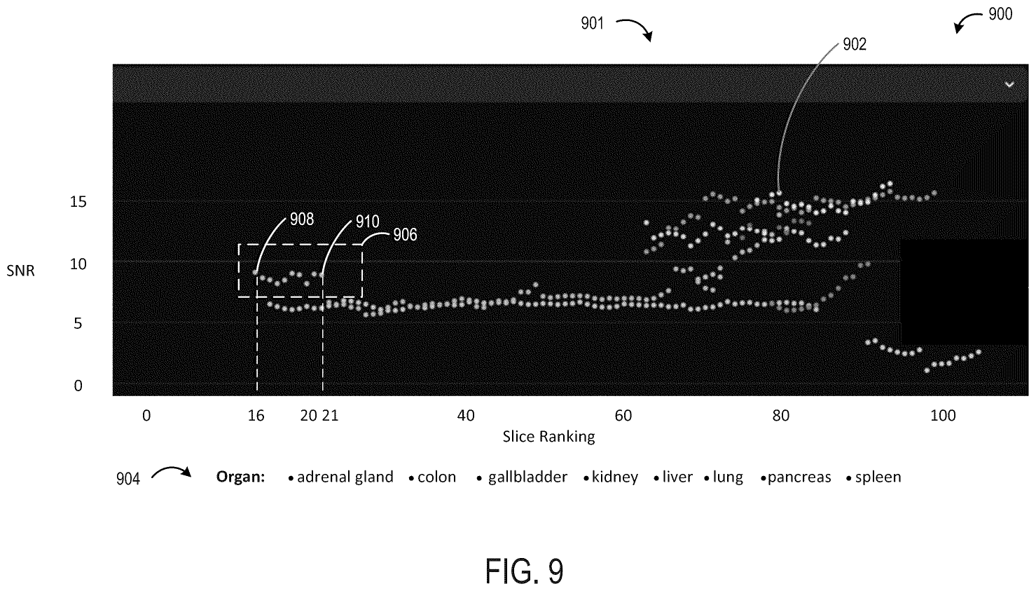

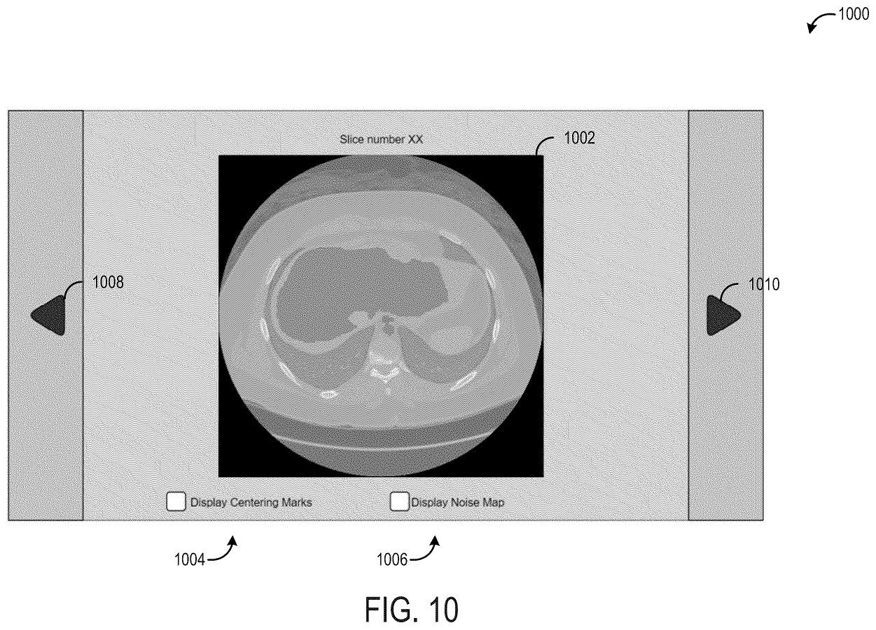

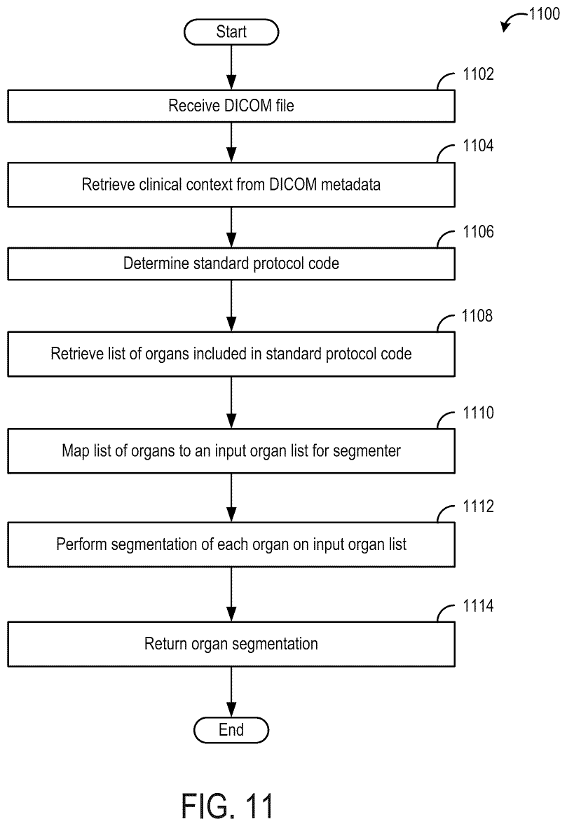

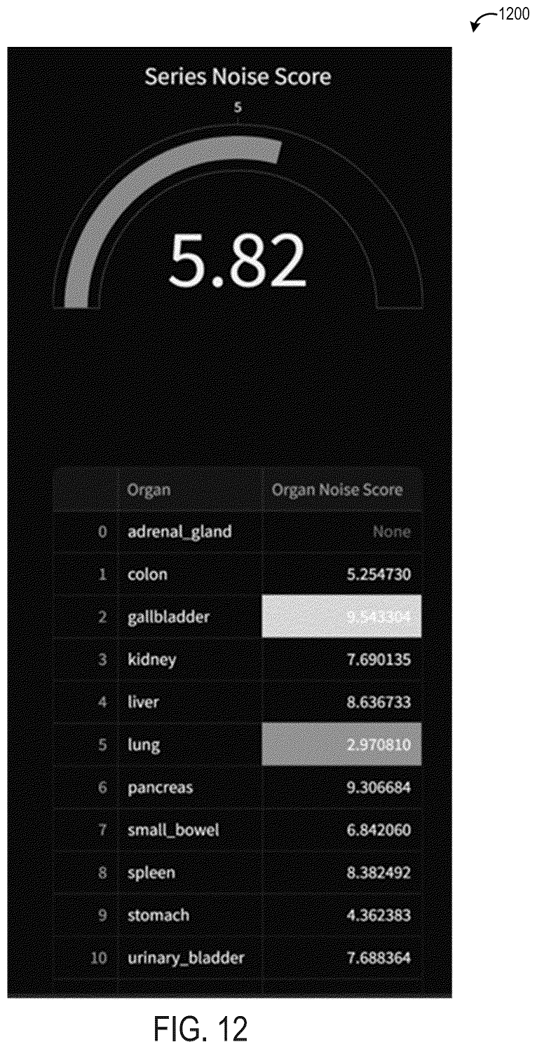

FIG. 1 shows an image quality assessment system, including an image quality scoring module that may be used to automatically score a quality of an image reconstructed from projection data acquired using a medical imaging system, in accordance with a strategy indicated in FIG. 2. The strategy may be implemented by following the general procedure shown in FIG. 3. The general procedure may be carried out by following one or more steps of a method shown in FIG. 4, which may include processing a set of DICOM files of a medical imaging exam, as described in relation to the method shown in FIG. 5; identifying and segmenting one or more organs and/or anatomical structures of a medical image of the set of DICOM files, as described in relation to the method shown in FIG. 11; and receiving feedback from a user of the image quality assessment system, as described in relation to the method shown in FIG. 6. An image quality score resulting from following the method may be displayed on a display device in an image quality GUI of the image quality assessment system shown in FIG. 7. The user may navigate through images shown in the GUI via a display panel such as the display panel shown in FIG. 8. The user may additionally view selectable, abbreviated representations of low-level metrics used to generate the image quality score in one or more display panels of the image quality GUI, as shown in FIG. 9. An individual 2-D slice image may be displayed, as shown in FIG. 10, for example, when the user selects a point on the graph of FIG. 9. Individual image quality scores for different organs over an image series may be displayed in a display panel shown in FIG. 12.

Referring to FIG. 1, a medical imaging system 100 is shown comprising an image quality assessment system 102, a medical imaging device 130, and a user 150, where user 150 may be a user of image quality assessment system 102, medical imaging device 130, or both image quality assessment system 102 and medical imaging device 130. In some embodiments, image quality assessment system 102 may be included in medical imaging device 130. For example, image quality assessment system 102 may be included in an image processing unit of medical imaging device 130, where the image processing unit is configured to reconstruct images of a subject using an iterative or analytic image reconstruction method, such as filtered back projection (FBP), advanced statistical iterative reconstruction (ASIR), conjugate gradient (CG), maximum likelihood expectation maximization (MLEM), model-based iterative reconstruction (MBIR), or a different reconstruction method. In other embodiments, image quality assessment system 102 may not be included in medical imaging device 130, and image quality assessment system 102 may be coupled to medical imaging device 130 via a wired or wireless connection, such that data may be transmitted between medical imaging device 130 and image quality assessment system 102.

In some embodiments, medical imaging device 130 either includes, or is coupled to, a picture archiving and communications system (PACS) 132. In an exemplary implementation, the PACS 132 is further coupled to a remote system such as a radiology department information system, hospital information system, and/or to an internal or external network (not shown) to allow operators at different locations to supply commands and parameters and/or gain access to the image data.

Image quality assessment system 102 may be a computing device, such as a desktop computer (e.g., a PC or a workstation), a laptop, a tablet, or different kind of computing device. Image quality assessment system 102 may be an image processing device dedicated to reviewing images from medical exams, such as an image review server, an image post-processing system, a PACS system (e.g., PACS 132), an acquisition console on an acquisition machine, or a cloud multi-tenant image review service.

Medical images acquired via medical imaging device 130 during a medical imaging exam may be stored in accordance with a Digital Imaging and Communications in Medicine (DICOM) standard, and image quality assessment system 102 may receive images from one or more DICOM files 140. The DICOM standard defines a file format that includes various fields for information that an image processing application may use to display and/or preprocess imaging data included in a DICOM file. DICOM files 140 may include raw image data 142 acquired during the medical imaging exam. DICOM files 140 may also include a plurality of description fields 144, where each description field 144 may include one or more expressions relevant to the medical imaging exam, an acquisition protocol of the exam, a series of the exam and/or images of the exam. The expressions may be single words, or multi-word expressions. For example, the expressions may include an anatomy of a patient; contrast information, including a contrast phase and a contrast agent; an acquisition gating; a laterality of raw image data 142; a pathology detected in raw image data 142; reconstruction filters used; a multi-energy indication; a weighting and/or pulse sequence, in the case of magnetic resonance (MR) images; and/or other options selected during the medical exam.

Image quality assessment system 102 may include a processor 104, a memory 106, and a display device 120. Processor 104 may control an operation of image quality assessment system 102, and may receive control signals from user 150 via display device 120. Display device 120 may include a display (e.g., screen or monitor) and/or other subsystems. In some embodiments, display device 120 may be integrated into image quality assessment system 102, where a user may interact with, adjust, or select control elements in the display device 120 (e.g., buttons, knobs, touchscreen elements, etc.) to send one or more control signals to the processor 104 from display device 120. In other embodiments, display device 120 is not integrated into the image quality assessment system 102, and the user may interact with, adjust, or select control elements in display device 120 via a user input device, such as a mouse, track ball, touchpad, etc., or the operator may interact with display device 120 via a separate touchscreen, where the operator touches a display screen of display device 120 to interact with image quality assessment system 102, or via another type of input device.

The operator may interact with image quality assessment system 102 via an image quality graphical user interface (GUI) 122. In some embodiments, image quality GUI 122 may be integrated into a GUI of a different system, such as medical imaging device 130. For example, various medical exam reviewing applications may be installed on medical imaging device 130 for viewing, reviewing, navigating through, and/or analyzing images of a medical exam. Each medical exam reviewing application may be suitable and/or preferred for a different type of medical exam. Each medical exam reviewing application may include a corresponding GUI for reviewing a medical exam, where image quality GUI 122 may be integrated into the corresponding GUI.

Processor 104 may execute instructions stored on the memory 106 to control image quality assessment system 102. Processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

As discussed herein, memory 106 may include any non-transitory computer readable medium in which programming instructions are stored. For the purposes of this disclosure, the term “tangible computer readable medium” is expressly defined to include any type of computer readable storage. The example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g. for extended period time periods, permanently, brief instances, for temporarily buffering, and/or for caching of the information). In some embodiments, the non-transitory computer readable medium may be distributed across various computers and/or servers (e.g., provided via web services). Computer memory of computer readable storage mediums as referenced herein may include volatile and non-volatile or removable and non-removable media for a storage of electronic-formatted information such as computer readable program instructions or modules of computer readable program instructions, data, etc. that may be stand-alone or as part of a computing device. Examples of computer memory may include any other medium which can be used to store the desired electronic format of information and which can be accessed by the processor or processors or at least a portion of a computing device. In various embodiments, memory 106 may include an SD memory card, an internal and/or external hard disk, USB memory device, or similar modular memory.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Memory 106 may store an image quality scoring module 108 that comprises instructions for generating an image quality score of a medical image, as disclosed herein. An overall procedure for generating the image quality score is shown in FIG. 3. Specifically, image quality scoring module 108 may include instructions that, when executed by processor 104, cause the image quality assessment system 102 to conduct one or more of the steps of method 400, as described in further detail below.

Image quality scoring module 108 may include various modules including instructions for performing various tasks involved in generating a quality score for the medical image. Image quality scoring module 108 may include a metadata ingestion module 110 that comprises instructions for ingesting image data from one or more DICOM files 140. The image data may include metadata stored in one or more of the description fields 144 of the one or more DICOM files 140. The ingestion of the image data performed with the metadata ingestion module 110 may include extracting and aggregating the metadata, as described in greater detail in reference to FIG. 5. In particular, metadata ingestion module 110 may include instructions that, when executed by processor 104, cause the image quality assessment system 102 to conduct one or more of the steps of method 500 of FIG. 5, as described in further detail below.

Image quality scoring module 108 may include a segmenter 111. Segmenter 111 may include segmentation software configured to detect and mark boundaries of one or more anatomical structures and/or organs in slices of a medical image included in the DICOM files 140. In various examples, segmenter 111 may include and rely on a deep learning model trained to perform segmentation of medical images. It should be appreciated that the term “organ”, as used herein, may alternatively refer to an anatomical structure that is not an organ, such as a bone or portion of a skeleton, joint, tendon, muscle group, etc. Segmenter 111 may include third-party commercial software.

Image quality scoring module 108 may include an image quality score generator 112. In various embodiments, image quality score generator 112 may include one or more ML models 114, which may be trained to generate (e.g., estimate) an image quality score for an image received from DICOM files 140. One advantage of using image data included in DICOM files for training the one or more ML models 114 is that the image data is independent of an acquisition system's vendor and/or model, and may be generated from various proprietary systems. As a result, it is possible to use image data from multiple systems and/or sites to train an ML model, whereby an increased amount of data may be collected in a shortened time frame. Generation of the image quality score is described in greater detail below in reference to FIG. 4. An example of a GUI including the image quality score is shown in FIG. 7.

FIG. 2 shows a general strategy diagram 200 for generating an image quality score for a medical image, such as a CT image reconstructed from projection data acquired via a CT system, using an image quality assessment system such as image quality assessment system 102 of FIG. 1. The general strategy depicted in FIG. 2 includes 3 stages. In a first stage 202, low-level metrics are calculated for each slice of the medical image, from a physical definition of noise, contrast, and texture. At a second stage 204, an image quality score for the medical image is generated from the low-level metrics by an image quality score generator (e.g., image quality score generator 112), using an ML model (e.g., an ML model 114). For example, the low-level metrics may be calculated for each slice of an organ and/or anatomical structure of the image, and the low-level metrics may be inputs into the ML model. An image quality score corresponding to the organ and/or anatomical structure may be an output of the ML model. The outputs of the ML model for each slice may be aggregated across the slices of the medical image to generate the image quality score. At a third stage 206, the image quality score of the organ and/or anatomical structure and the low-level metrics used to generate the image quality score are displayed to a user of the image quality assessment system. Using a GUI of the CT system, the user may provide feedback regarding the image quality score, which may be collected, stored, and used to refine or retrain the one or more ML models to increase an accuracy of the image quality score.

An additional advantage of the methods and systems described herein is that the user can select a portion of the medical image, (e.g., the desired anatomical structure or organ), and view an image quality score for the selected portion, which may be different from the image quality score for the entire image and/or image quality scores of other portions of the medical image. The portion may be selected by the user via the GUI. For example, boundaries of various organs in the medical image may be indicated, and an organ of the various organs may be selected via a user input device such as a mouse. When the organ is selected, low-level metrics of the organ may be analyzed to generate a corresponding image quality score for the organ. Additionally or alternatively, an organ in the medical image may be selected from a menu or list of the various organs, or in a different manner.

In some examples, a plurality of organs and/or anatomical structures may be selected by the user, and low-level metrics corresponding to each organ or anatomical structure of the plurality of organs and/or anatomical structures may be aggregated to generate an aggregate image quality score for the plurality of organs or anatomical structures. In other words, a first selected portion or portions of the medical image may result in a first image quality score based on low-level metrics calculated (and aggregated) from the selected portion or portions, where the first image quality score is not based on low-level metrics calculated from slices and/or areas of the medical image outside the first selected portion or portions. The user may select a second portion or portions of the medical image, to generate a second, different image quality score based on a different set of low-level metrics calculated (and aggregated) from the second portion or portions, where the second image quality score is not based on low-level metrics calculated on slices/portions outside the selected portion or portions.

When the user deselects the first portion of the medical image and selects the second portion of the medical image, the image quality score generator may not be relaunched to recalculate the image quality score for the second portion. Rather, slices included within the selected portion are selected, and the image quality score for selected portion may be calculated by aggregating the image quality scores generated for the selected portion at each individual slice of the selected slices. In this way, image quality scores for different selected portions may be displayed in the GUI more rapidly than if the image quality score generator were relaunched each time a portion of the medical image is reselected by the user. As a result, in an amount of time available to the user to view the medical image, the user may provide feedback with respect to the image quality score displayed for the medical image, and additionally specify one or more portions (e.g., organs and/or anatomical structures) of the medical image, view image quality scores corresponding specifically to the portions, and provide feedback on the image quality scores of the portions. By reducing an amount of time taken by the user to provide feedback on the medical image and/or the portions of the medical image, an amount of feedback data collected by the image quality assessment system may be increased. By increasing the amount of feedback data collected by the image quality assessment system, an amount of training data used to retrain the ML model may be increased, thereby increasing the accuracy and/or performance of the ML model. Thus, a virtuous cycle may be created, leading to increasingly accurate image quality scores and as a result, higher quality images.

FIG. 3 shows an overview of stages of a high-level procedure 300 for generating the medical image quality score within the image quality assessment system. The stages illustrated in FIG. 3 are described in greater detail below in reference to the methods of FIGS. 4, 5, 6, and 11.

In a first stage of high-level procedure 300, DICOM series data 302 is ingested into the image quality assessment system during a data ingestion process 304, to generate an image volume including a plurality of slices. During ingestion, metadata included in DICOM series data 302 may be extracted and aggregated, as described below in reference to FIG. 5.

In a second stage of high-level procedure 300, an organ segmentation process 306 may then be performed on each slice of the image volume. During the organ segmentation process 306, a list of one or more organs of the image volume may be retrieved from a protocol of the DICOM series. The one or more organs may be segmented within each slice of the image volume using a segmenter (e.g., segmenter 111), as described below in reference to FIG. 11.

In a third stage of high-level procedure 300, each segmented organ may be processed across a set of respective slices including the organ to determine a set of image quality scores for the one or more organs during an image quality score generation process 308, as described in detail below in reference to FIG. 4. The segmentation of the organs ensures that the image quality score of the organ is calculated based on image data inside a boundary of the organ in each slice, and not image data outside the boundary of the organ.

In a fourth stage, during an image quality score aggregation process 310, the set of image quality scores for each organ may be aggregated to generate an overall image quality score for the image, and the aggregated results may be displayed to a user of the image quality assessment system at an interactive result display step 312, as described in detail below in reference to FIG. 6.

FIG. 4 shows an exemplary method 400 for generating and displaying a quality score for an image, such as a CT image, based on the procedure described above in reference to FIG. 3. In various embodiments, the image may be stored in a DICOM file (e.g., the DICOM series data 302 of FIG. 3). Method 400 and other methods described herein are described with reference to an image quality assessment system, such as image quality assessment system 102 of FIG. 1, and in particular, an image quality scoring module (e.g., IQ scoring module 108) of the image quality assessment system. Method 400 and the other methods described herein may be implemented via computer-readable instructions stored in a memory of an image quality assessment system, and executed by a processor of the image quality assessment system, such as memory 106 and processor 104 of image quality assessment system 102 of FIG. 1. In some examples, the image may be a 3D image and thus may be comprised of image data from a plurality of 2D slices. In other examples, method 400 may be applied to a volume of image data comprised of a plurality of 2D slices.

Method 400 begins at 402, where method 400 includes ingesting a DICOM file corresponding to the image (e.g., the DICOM file that includes the image). Ingestion of the DICOM file is described in greater detail in FIG. 5.

Referring to FIG. 5, an exemplary method 500 is shown for ingesting and processing a DICOM file, such as a DICOM file 140 of FIG. 1, via an image quality assessment system (e.g., medical imaging system 100). Method 500 begins at 502, where method 500 includes receiving a DICOM file. In some embodiments, the DICOM file may be received from a CT system (e.g., medical imaging device 130) electronically coupled to the image quality assessment system, where the DICOM file includes a plurality of image acquired by the CT system. The DICOM file may be received at a time of a scan performed by an operator of the CT system, or the DICOM file may be received after a scan is performed.

At 504, method 500 includes extracting metadata from the DICOM file. In various embodiments, the metadata may be extracted and aggregated by level (e.g., study, series, image, etc.) For example, in a first step, metadata pertaining to a study of the DICOM file may be extracted from one or more description fields (e.g., description fields 144 of FIG. 1) of the DICOM file. In a second step, metadata pertaining to a series of the DICOM file may be extracted from the one or more description fields. In a third step, metadata pertaining to an image of the DICOM file may be extracted from the one or more description fields, and so on.

At 506, method 500 includes aggregating the extracted metadata by level. At 508, aggregating the extracted metadata by level includes aggregating the metadata collected with respect to a study of the DICOM file. At 510, aggregating the extracted metadata by level includes aggregating the metadata collected with respect to a series of the DICOM file. At 512, aggregating the extracted metadata by level includes aggregating the metadata collected with respect to an image included in the DICOM file. The aggregated metadata may include a list of organs (and/or anatomical structures) that are visible in the slices of the image data. In some examples, ingestion and processing of the DICOM file may include ingestion of pixel data (e.g., images) in the DICOM file. In some examples, the DICOM file may include a plurality of images (e.g., series images) and only a subset of the plurality of images may be ingested, which may avoid ingesting and storing each image of the DICOM file. Further, information in the DICOM file other than the metadata and sampled pixel data may not be ingested. Method 500 ends.

Returning to FIG. 4, at 404, method 400 includes determining the boundaries of one or more organs in each slice of the image and generating masks of the organs based on the boundaries. The boundaries may be determined using segmentation software known in the art (e.g., segmenter 111 of FIG. 1). The segmentation software may output a mask for each slice of the image, where the mask is a two-dimensional array of ones (corresponding to image data inside the boundaries of a respective organ) and zeros (corresponding to image data outside the boundaries of the respective organ). The boundaries of each selected organ may be used to distinguish image data corresponding to the selected organ from image data not corresponding to (e.g., outside of) the selected organ. The automatic detection and segmentation of the one or more organs or anatomical structures is described in greater detail below in reference to FIG. 11.

The organs and structures may be segmented based on sampled data. In other words, rather than using all or most of the images in a given series to segment the organs and structures, a portion of the images of a DICOM series dataset (with a uniform distribution) may be selected and used, to reduce storage and costs of computing. For example, a sample of 100 images may be taken from a total number of 700 images of an image series. As explained above, in some examples, the sampling may be performed when the DICOM file is ingested.

At 406, method 400 includes calculating low-level metrics to evaluate noise, texture, and contrast in each slice for each detected organ and/or anatomical structure, using the organ masks to exclude image data outside the boundaries of the organs. The sampled data used for the organ segmentation may additionally be used to calculate the low-level metrics and image quality scores of the detected organs and/or anatomical structures that are aggregated to generate the overall image quality score. Calculating the low-level metrics may include, for example, calculating an NPS for each structure/organ. The computation of the NPS is made possible due to the segmentation of the organs and structures, which results in uniform raw data. In other words, an advantage of the proposed image quality assessment system is that an accuracy of the NPS calculation, and the calculation of other low-level metrics, is increased with respect to an alternative system where image quality is determined for a whole image or a portion of the whole image, where the image quality score may be determined based on image data from areas of interest (e.g., organs, anatomical structures, etc.) and also image data from areas that are not of interest to the user (e.g., not included in the organs and anatomical structures).

From a two-dimensional (2D) NPS curve, an area under the curve may be calculated and used as an image quality metric. Additionally, an overall noise level and/or SNR may be computed which may be further broken down into one or more noise levels and/or SNRs of the detected organ; an overall CNR, which may be further broken down into one or more CNRs of the detected organ; a uniformity metric; a noise level; and so on. It should be appreciated that the examples provided herein are for illustrative purposes, and other low-level metrics may be included without departing from the scope of this disclosure.

The low-level metrics may be calculated with the same formulas across various clinical contexts and hospitals, but the low-level metrics and noise scores may be normalized based on a clinical context from a same hospital to facilitate a comparison of image quality scores between images acquired within the hospital. In other examples, for example in a cloud context, where data from different hospitals may be compared, the noise scores may be computed and normalized across different hospitals, clinical contexts, region, country, patient profile, etc.

At 408, method 400 includes generating image quality scores for each slice including each organ based on the calculated low-level metrics, using an ML model (e.g., an ML model 114). For example, a plurality of numerical values obtained from the calculated low-level metrics may be inputs into the ML model, and the ML model may be trained to predict an image quality score based on the plurality of numerical values. In various embodiments, the ML model is trained using training data including low-level metrics of training images, and ground truth image quality scores of the training images determined by human experts. In other embodiments, different training data may be used. An image quality score may be generated for each detected organ and/or anatomical structure.

At 410, method 400 includes aggregating the image quality scores and low-level metrics of the slices including each organ and/or anatomical structure, to generate image quality scores and aggregated metrics for the organs. For example, aggregating the image quality scores of the slices included in each organ and/or anatomical structure may include calculating an average of the image quality scores of the slices, or a different function may be used to aggregate the image quality scores. Similarly, the low-level metrics used to generate the image quality scores for the slices of an organ and/or anatomical structure may be aggregated, such that a single numerical value corresponding to a low-level metric may be obtained for the organ. The single numerical value may be normalized between zero and 10, based on a desired scope of the clinical context (e.g., based on data from a same hospital, from a same hospital group, from a same type of patient, etc.) The normalized single numerical value may be displayed in the GUI, so that a user can review the image quality score and assess its accuracy.

At 412, method 400 includes aggregating the image quality scores of the organs and/or anatomical structures, to generate an overall image quality score for the image. In various embodiments, aggregating the image quality scores of the organs may include calculating an average of the image quality scores of the organs, or a different function may be used to aggregate the image quality scores. Additionally, the image quality scores of each image in a series may be aggregated to generate an overall image quality score for the series.

At 414, method 400 includes displaying the image and the overall image/series quality score in a GUI of the image quality assessment system (e.g., image quality GUI 122). The image may include the detected organs and/or anatomical structures, which may be selectable in the image. Displaying the image quality score is described in greater detail below in reference to FIG. 6. At 416, method 400 includes receiving image quality feedback from a user of the image quality assessment system, and method 600 ends.

As an example of how method 600 might be applied, an operator of a CT system may perform a CT scan on a patient. A 3-D CT image acquired by the CT system may be stored in a DICOM file in a memory of the CT system, or in a different system coupled to the CT system. A radiologist may wish to view the 3-D CT image, for example, to perform a diagnosis of the patient. The radiologist may load the DICOM file into the image quality assessment system to view the 3-D CT image. The image quality assessment system may ingest the DICOM file, and extract metadata associated with the 3-D CT image. The image quality assessment system may determine boundaries of each organ of a set of organs included in the 3-D CT image, using segmentation software (e.g., segmenter 111).

For each 2-D slice of the 3-D CT image, low-level image quality metrics may be calculated that may be indicative of a quality of the 2-D slice. Additionally, low-level image quality metrics may be calculated for the segmented organs included within the 2-D slice, which may be indicative of a quality of portions of the 2-D slice corresponding to the organs. Results of the low-level image quality metrics for the 2-D slice may be inputted into an ML model of an image quality score generator, which may generate an image quality score of the 2-D slice. Additionally, results of the low-level image quality metrics for the segmented organs may be inputted into the ML model to generate image quality scores for the portions of the 2-D slice corresponding to the identified organs.

Image quality scores for the organs may be calculated, where the image quality score of an organ may be a function of (e.g., an average of) the image quality scores of each of the 2-D slices associated with the organ during the slice reconciliation. Additionally, the results of the low-level image quality metrics used to generate the image quality scores of each of the 2-D slices associated with the organ may be aggregated across the 2-D slices to generate aggregate (e.g. average) low-level metric data for the organs.

The image quality assessment system may then generate an overall image quality score for the 3-D CT image, as a function of the image quality scores of the 2-D slices included in the 3-D CT image. Additionally or alternatively, the overall image quality score may be generated as a function of the image quality scores of the organs.

The image quality assessment system may display the 3-D CT image and/or the 2-D slices in a GUI of the image quality assessment system, to be viewed by the radiologist. For each image the radiologist views, the image quality assessment system may display the overall image score associated with the image. In various embodiments, the overall image score may be displayed in an interactive graphical element, as described below in reference to FIG. 7. If the radiologist disagrees with an image quality score assigned to an image or to an organ of the image, the radiologist may adjust the image quality score to a more suitable image quality score, using the interactive graphical element. For example, if the radiologist feels that the image quality score is too high, the radiologist may reduce the image quality score, and if the radiologist feels that the image quality scores too low, the radiologist may increase the image quality score. When the image quality score is adjusted, the adjusted image quality score may be saved by the image quality assessment system. The saved, adjusted image quality score may be included in training data of the ML model, where the ML model may be retrained or refined in a subsequent training stage using the training data. In this way, an accurate set of ground truth training data based on human expert opinions may be obtained in an efficient manner, for example, from a plurality of radiologists, thereby increasing in accuracy and/or a performance of the ML model.

Referring now to FIG. 6, an exemplary method 600 is shown for displaying image quality scores associated with a medical image to a user of an image quality assessment system, such as image quality assessment system 102 of FIG. 1, in a GUI of the image quality assessment system, and receiving feedback from the user regarding an accuracy of the image quality score. The image quality scores may be generated via a method such as method 400 described above in reference to FIG. 4.

Method 600 begins at 602, where method 600 includes displaying the medical image and an image quality score generated for the medical image on a display device (e.g., display device 120 of FIG. 1). As described above, the image quality score may be an aggregate score that aggregates image quality scores generated from slices of the medical image. Additionally, images of the one or more organs and/or anatomical structures of the medical image and the image quality scores and low-level image quality metric data associated with the organs and/or anatomical structures may be displayed and updated when different views or organs are selected by the user, without regenerating the image quality scores of the slices, by selecting control elements in an image quality GUI, such as the image quality GUI shown in FIG. 7.

Turning briefly to FIG. 7, an image quality GUI 700 is shown of an image quality assessment system, such as image quality GUI 122 of image quality assessment system 102. Image quality GUI 700 includes a reference image 702, where reference image 702 is a 2-D image of a scanned subject showing a series scanned area of a 3-D medical image (also referred to herein as the medical image) acquired via a medical imaging system (e.g., medical imaging device 130 of FIG. 1). Image quality GUI 700 includes an image quality score 710 indicating a quality of the medical image, specifically, to an overall quality of slices of the medical image included in the series scanned area. In various embodiments, image quality score 710 is a numeric value calculated as described above in reference to FIG. 4. Image quality score 710 may be displayed within a textual context, as shown in image quality GUI 700, or image quality score 710 may be displayed in a different manner, such as within a graphical element of image quality GUI 700.

Image quality GUI 700 includes an image quality score indicator 720, on which image quality score 710 is indicated. In the embodiment depicted in FIG. 7, image quality score indicator 720 is a slider 722 showing a range of image quality score descriptors (e.g., perfect, good, ok, bad, etc.), where the image quality score is indicated by a needle 724 of slider 722. In other embodiments, a different graphical element may be used to indicate the image quality score 710, such as, for example, a dial.

Image quality score indicator 720 may be an interactive graphical element, where a user of the GUI may adjust a relative position of needle 724 within slider 722 to change image quality score 710. For example, the user may not agree with image quality score, and the user may wish to adjust the image quality score to a more accurate image quality score. For example, the user may feel that the image quality is higher than indicated by the image quality score, and the user may adjust needle 724 upward in a direction 752 to increase the image quality score, or the user may feel that the image quality is lower than indicated by the image quality score, and the user may adjust needle 724 downward in a direction 750 to decrease the image quality score. In various embodiments, when the user adjusts the image quality score in image quality score indicator 720, the adjusted image quality score is stored in a memory of the image quality assessment system (e.g., memory 106). The adjusted image quality score may be included in training data used to retrain an ML model used to generate the image quality score (e.g., at a later stage), to increase an accuracy of the image quality score.

Image quality GUI 700 may include a menu 726, which may include, as menu items, a limited list of low-level metrics used to generate image quality score 710. For example, the limited list of low-level metrics may include an SNR, a CNR, an NPS, etc. In response to the user selecting a menu item corresponding to a low-level metric of the limited list of low-level metrics, one or more summaries of results of the low-level image quality metric may be displayed in a display panel (e.g., a pop-up) of the GUI. The one or more summaries may include tables of low-level image quality metric results, graphs of low-level image quality metric results, and/or images of slices of the medical image. Examples of the one or more summaries are shown below in FIGS. 8, 9, and 10.

One or more organs and/or anatomical structures may be identified in reference image 702. In the depicted example, the one or more organs and/or anatomical structures include at least a spinal column 704, an upper intestine 706, a lower intestine 708, and a kidney 712. The one or more organs may be distinguished from other elements of the medical image via boundaries detected during a prior segmentation process. Areas of reference image 702 corresponding to the identified organs, meaning, within the boundaries, may be selectable by the user. For example, the user may click on spinal column 704 in reference image 702 using a mouse or other user input device, and spinal column 704 may be selected. When spinal column 704 is selected, spinal column 704 may be highlighted in reference image 702. When spinal column 704 is selected, image quality score 710 may be updated in GUI 700 to an image quality score of spinal column 704, which may be different from a previously displayed image quality score for reference image 702. The user may adjust the image quality score of spinal column 704 using slider 722. The user may then select a different organ in reference image 702, to view the image quality score of the different organ.

Additionally or alternatively, a list of the organs identified in the medical image may be displayed in a menu 728. Using menu 728, the user can select one or more organs, and the selected organ may be highlighted in reference image 702. An advantage of using menu 728 to select an organ is that an organ may be selected that is obscured by other anatomical structures in reference image 702. In other examples, the user may select one or more organs in reference image 702 in a different manner.

Returning to FIG. 6, at 604, method 600 may include displaying low-level metrics and slice details. The low-level metrics and slice details may be displayed via display panels, such as the exemplary display panels shown in FIGS. 8, 9, and 10, and 12.

FIG. 8 shows an exemplary display panel 800, which may be displayed within GUI 700 of FIG. 7. Display panel 800 may be used by a user of the image quality assessment system to navigate to desired images included in one or more studies generated using a medical imaging system such as medical imaging system 100 of FIG. 1. For example, display panel 800 may be displayed when GUI 700 is launched. The user may select a desired image series to view using display panel 800.

Display panel 800 may include a list of studies, presented as rows in a table-based display. The table-based display may include a plurality of columns. For example, in the depicted embodiment, a first column 802 shows a list of studies; a second column 804 shows a list of series included in each study of the list of studies; a third column 806 shows a study date of each study; a fourth column 808 shows an image quality of each series, based on an image quality score generated in accordance with method 400 of FIG. 4; a fifth column 810 shows a RadLex Protocol ID (RPID) code associated with each series; a sixth column 812 shows a text description of each series; and a seventh column 814 shows a name of a patient of the study. Each row of display panel 800 may be selectable by the user. When the user selects a row, image quality information and scores may be presented for image data corresponding to the row (e.g., in GUI 700). The image quality information and scores may include SNR, CNR, NPS, and/or other charts. The SNR, CNR, NPS, and/or other data may be computed based on organs and anatomical structures detected within the images, and not based on image data outside the detected organs.

As an example of how display panel 800 may be used, the user may review the list of series shown in second column 804. The user may select a row to view a desired series, based on the RPID and/or series description, or based on an image quality associated with the series. For example, the user may wish to confirm the image quality, or adjust the image quality if deemed incorrect by the user. When the user selects a row, the user may select (for example, as a right-click option of a mouse) to view images included in the selected series. The images included in the selected series may be displayed in GUI 700. Using GUI 700, the user may review and adjust recorded image quality scores associated with the images.

FIG. 9 shows a display panel 900, which displays an exemplary overview of low-level metrics across a plurality of slices of a medical image. Display panel 900 shows a plot 901 of image quality data, where a vertical axis indicates a range of image quality values, and a horizontal axis indicates a progression (e.g., ranking) of slices of the medical image. A plurality of points 902 on plot 901 may show various organs included in a respective slice of the progression of slices. That is, each point 902 may represent an organ in the medical image, where a horizontal position of the point 902 indicates a slice in which the organ appears. In the depicted example, the image quality values are SNR values; in other examples, the image quality values may be CNR, NPS, or a different low level metric. Thus, by viewing the plot, a user can see a summary of how SNR (or a different low-level metric) varies for each organ in the medical image over various slices of the medical image. The points 902 may be color coded to differentiate between different organs, as indicated by legend 904.

For example, a dashed box 906 may include a set of points 902 corresponding to a gall bladder of a patient, which may all share the same color that is different from the colors of other organs in display panel 900. A first point 908 indicates an SNR of the gall bladder calculated for a first slice including the gall bladder, which has a slice ranking (e.g., position in the medical image) of 16. A second point 910 indicates an SNR of the gall bladder calculated for a last slice of the medical image including the gall bladder, which has a slice ranking of 21. Between slices 16 and 21, various other points indicate relative (e.g., normalized) SNRs of the gall bladder. Thus, the user can estimate a quality of the image of the gall bladder (with respect to other organs, and/or pre-established quality baselines) in individual slices and across all slices in the medical image.

In various embodiments, display panel 900 may be displayed as a result of the user selecting elements of data displayed on the display panels 800 and/or 850. For example, the user may select the low-level metric “SNR” in display panel 800, and in response, the image quality assessment system may generate a display panel 900 displaying the low-level image quality metric SNR for slices of the image.

Display panel 900 may be an interactive graph, and points 902 of the graph may be selectable by a user. When the user selects a point on the graph, the corresponding slice may be displayed in the GUI. An example of a slice corresponding to a point is shown in FIG. 10.

FIG. 10 shows an exemplary display panel 1000 showing a 2-D slice 1002 of a medical image. In various embodiments, 2-D slice 1002 may be displayed as a result of a user of an image quality assessment system (e.g., image quality assessment system 102 of FIG. 1) selecting a point corresponding to the slice in a graphical element displayed to the user showing details with respect to a quality of the medical image. For example, display panel 1000 may be displayed as a result of the user selecting a point (e.g., point 902) of interactive display panel 900 of FIG. 9. Display panel 1000 may include one or more control elements, such as control elements 1004 and 1006, which may be used to change in appearance of 2-D slice 1002. For example, the user may select control element 1004 to view centering marks of 2-D slice 1002, which may be superimposed upon 2-D slice 1002. The centering marks may indicate a uniformity of 2-D slice 1002. If a scanned subject of the medical image is not centered properly, a noise profile of the medical image may be affected, and the quality of the medical image may be deteriorated. Similarly, the user may select control element 1006 to view a noise map of 2-D slice 1002, which may be superimposed upon 2-D slice 1002. In this way, the user may view 2-D slice 1002 with or without the centering marks and the noise map. Graphical the 1000 may include directional arrow elements 1008 and 1010, which may be used to advance through a plurality of slices of the medical image. For example, the user may select directional arrow element 1010 to view a slice adjacent to 2-D slice 1002 in a first direction of the medical image, and the user may select directional arrow element 1008 to view a slice adjacent to 2-D slice 1002 and a second, opposing direction of the medical image.

FIG. 12 shows another exemplary display panel 1200 that may be generated by an image quality assessment system such as image quality assessment system 102 of FIG. 1. Display panel 1200 shows a plurality of scores calculated for each organ of a plurality of organs included in series of medical images processed using the image quality assessment system. Each score may be calculated based on individual low-level quality metrics (e.g., SNR, CNR, etc.), a combination of low-level quality metrics, or individual image quality scores that are calculated for each organ based on the low-level metrics, over a plurality of slices of each medical image in the series, in accordance with the methods described herein. For example, in the depicted example, a noise score may be generated for each identified organ that is based on a combination of low-level noise metrics. Thus, FIG. 12 shows an overall summarization of the image qualities of each organ in a series. Scores that are outside of an expected range of values may be highlighted in display panel 1200.

Thus, when the user views an image score of a medical image on a display device of the image quality assessment system, the low-level metric data used to generate the image score may be viewed in various selectable abbreviated representations via summary display panels that display select low-level metric data, without the user having to view comprehensive low-level metric data, which may entail the user having to navigate or scroll through a large number of results, many of which that are not of interest to the user. Rather, the low-level metrics are advantageously displayed in a manner in which the user may view summarized image quality data at various levels of detail and with respect to various organs or anatomical structures in the medical image, where the user may select elements of data in a first summary display panel to view additional image quality data in a second summary display panel, select elements of data in the second summary display panel to view additional image quality data in a third summary display panel, and so on. When the user selects an element of data to display a summary display panel, the summarized image quality data may be displayed quickly and responsively, as the image quality score generator does not recalculate the low-level metrics or the image quality scores in response to user input.

Returning to FIG. 6, at 606, method 600 includes determining whether an organ or anatomical structure in the medical image has been selected by the user, as described above in reference to FIG. 7. If at 606 it is determined that an organ has not been selected, method 600 proceeds to 608. At 608, method 600 includes continuing to display the image quality score and the low-level metrics in the GUI. Alternatively, if at 606 it is determined that one or more organs have been selected, method 600 proceeds to 610.

At 610, method 600 includes updating the image quality score and/or low-level metrics based on the selected organ. In other words, when an organ is selected by the user, a number of slices over which the low-level metrics are aggregated to produce the image quality score for the organ, and boundaries of the slices corresponding to the organs and within which the low-level metrics are calculated may change. As a result of the change, the image quality score will be adjusted. As the image quality score is adjusted, a position of needle 724 may be adjusted in a corresponding direction. For example, if a first organ is selected that has a first image quality score that is greater than an overall image quality score of the medical image, needle 724 may be adjusted in direction 752. Alternatively, if a second organ is selected that has a second image quality score that is less than the overall image quality score of the medical image, needle 724 may be adjusted in direction 750.

The results of the low-level metrics are adjusted accordingly. As different organs are selected and as the number of slices used in the calculation of the image quality score changes, the aggregation of the low-level metrics also changes. For example, a first image quality score may be generated based on a first number of slices, where the first number of slices corresponds to a first organ. The user may subsequently select a second organ of interest in the image. A second image quality score may be generated based on a second number of slices, where the second number slices corresponds to the second organ. The first image quality score may be based on a first set of low-level metrics aggregated over the first number of slices, and the second image quality score may be based on a second set of low-level metrics aggregated over the second number of slices.

Additionally, when viewing a medical image, the user may select one or more menu items of menu 726, and as a result, a first summary of a first set of selected low-level metrics aggregated over the medical image may be displayed in the GUI. The user may then select one or more different menu items of menu 726, and as a result, a second summary of a different, second set of selected low-level metrics aggregated over the second number slices may be displayed at the GUI. The summaries may be advantageously displayed while the image quality score generator is in an unlaunched state. In other words, the generation of the image quality scores may occur prior to the user making selections via the GUI, and the calculation of the aggregated image quality scores may occur in response to the selection of the organs by the user, without the image quality score generator calculating new image quality scores using the ML model. As a result, the summaries and/or an updated image quality score may be displayed in the GUI in a rapid and responsive fashion, which may encourage the user to provide feedback.

At 612, method 600 includes determining whether the image quality score has been adjusted by the user (e.g., by adjusting a position of needle 724 within slider 722). If at 612 it is determined that image quality score has not been adjusted by the user, method 600 proceeds to 614. At 614, method 600 includes continuing to display the image quality score and the low-level metrics on the display device. Alternatively, if at 612 it is determined that the image quality score has been adjusted by the user, method 600 proceeds to 616.

At 616, method 600 includes storing the adjusted image quality in a memory of the image processing device (e.g., memory 106). In some embodiments, additionally or alternatively, the low-level metrics used to generate the adjusted image quality score and displayed in the GUI may be stored in the memory. Method 600 ends.

Referring now to FIG. 11, a method 1100 is shown for identifying and segmenting one or more organs in a medical image volume, so that individual image quality scores can be calculated for the one or more organs. Method 1100 may be performed by an image quality assessment system, such as image quality assessment system 102 of FIG. 1. Method 1100 may be performed as part of method 400 of FIG. 4.

At 1102, method 1100 includes receiving a DICOM file (e.g., DICOM files 140) including a medical image volume comprising a plurality of slices. The DICOM file may be including in the DICOM series data ingested by the image quality assessment system as described in method 400. The DICOM file may also include a localizer image corresponding to the medical image volume.

At 1104, method 1100 includes retrieving a clinical context from metadata stored in one or more of the description fields of the DICOM file. The clinical context may include at least a protocol name or code used for generating the medical image volume and a description of an image series of the medical image volume. In some examples, the clinical context may be connected to other data sources, such as an entry in an electronic health record (HER) or Radiology Information System (RIS) of a patient of the medical image volume. The clinical context is most often “richer” in information than the protocol name, and the more information that can be obtained regarding which organs are targeted, the more “focused” the IQ score can be. It should be appreciated that the radiologist will see the clinical context when examining the images and viewing the image quality scores.

As a first example, a first protocol may have a protocol name “CT Angiography (CTA) Protocol”. A first clinical context retrieved for this protocol name may be “Pulmonary Embolism”, as this protocol may be used to evaluate the pulmonary arteries in lungs to detect blood clots. A second clinical context retrieved for this protocol name may be “Aortic Dissection”, as this protocol may be used to assess an aorta for tears or dissections. As a second example, a second protocol may have a protocol name “CT ABD-PEL with IV Contrast (Portal Veinous Phase)”. A first clinical context retrieved for this protocol name may be “Evaluation of the pancreas for suspected pancreatic”. A second clinical context retrieved for this protocol name may be “Evaluation of the bowel for suspected inflammatory bowel disease”.

At 1106, method 1100 includes determining a standard protocol code associated with the retrieved protocol name or code. That is, the retrieved protocol names included in various DICOM files may differ, and the standard protocol code may be used to resolve differences between the various DICOM files to a common reference. The standard protocol code may be a protocol code of a publicly available radiology lexicon. In various examples, the standard protocol code may be a RadLex protocol code of the RadLex lexicon. However, it should be appreciated that in some examples, a different protocol standard not based on the RadLex lexicon may be used. For example, a Logical Observation Identifiers Names and Codes (LOINC) standard protocol code may be used.

At 1108, method 1100 includes retrieving a list of organs included in the medical image volume from the RadLex lexicon, based on the standard RadLex protocol code. That is, the RadLex lexicon may specify the organs and/or anatomical structures included in images acquired from a CT scan based on the standard RadLex protocol code.

At 1110, method 1100 includes, for a set of organs included in the list of organs, mapping a first name of each organ retrieved from the RadLex lexicon to a second name of the organ used by segmentation software used to segment the organ in the medical image volume. That is, the segmentation software (e.g., segmenter 111 of FIG. 1) may take as input a name of an organ or anatomical structure to be segmented that is different from a reference name in the RadLex lexicon and/or other accepted names of the organ or anatomical structure.

For example, a RadLex reference name may be, “CT ABDOMEN PANCREAS WITH IV CONTRAST” (RPID947). However, the segmentation software may have to mention of the scanning technique, only the organ. Therefore, for the above RadLex reference name, the corresponding term relied on by the segmentation software may be simply “pancreas”.

The mapping of the first name of each organ retrieved from the RadLex lexicon to the second name of the organ used by segmentation software may be performed using a lookup table generated in advance. The lookup table may be generated manually. In some examples, the lookup table may be generated with the aid of a large language model (LLM), where the RadLex lexicon is added as context. For example, a single system prompt may be entered into the LLM that takes the list of target labels used by the segmentation software, and requests that an input RadLex label provided by the user be assigned a most relevant label of the target labels. A user prompt provides the input (RadLex) label to classify, and an API of the LLM (with system prompt and user prompt) may be called for each input label in a loop over the list of possible target labels for inputting into the segmentation software.

At 1112, method 1100 includes performing a segmentation of each organ included in the set of organs on the list of organs. The segmentation may be performed using segmentation software (e.g., segmenter 111) including a deep learning model. At 1114, method 1100 includes returning the organ segmentations generated by the segmentation software. The organ segmentations may be two-dimensional binary masks, where zeros in the masks indicate image data outside a boundary of an organ, and ones in the masks indicate image data inside the boundary. Method 11 ends.

Thus, systems and methods are disclosed herein for automatically generating an overall image quality score for a medical image, such as a CT image acquired via a CT imaging system, via an image quality assessment system including an image quality score generator. The overall image quality score may be calculated by the image quality score generator based on a plurality of low-level metrics, using an ML model trained on training data including image quality scores and/or low-level metrics as ground truth data. The low-level metrics may be calculated for a plurality of anatomical structures and/or organs included in the medical image. Boundaries of the anatomical structures and/or organs may be determined using segmentation software, and the boundaries may be used to generate organ masks that isolate image data within the anatomical structures and/or organs for assessment and exclude image data outside the anatomical structures and/or organs. The low-level metrics for each organ may be calculated using the organ masks. The overall image quality score may be generated by aggregating image quality scores calculated for slices of the medical image associated with each organ, and/or aggregating image quality scores calculated for specific organs and/or anatomical structures of the medical image. The overall image quality score may be displayed in a GUI of the image processing device, within an interactive graphical element that allows a user to provide image quality feedback on the medical image by adjusting the overall image quality score in the GUI. The feedback (e.g., the adjusted overall image quality score) may be stored in a memory of the image quality assessment system, and used to retrain the ML model to increase an accuracy or performance of the ML model.

Additionally, since the image quality score is adjusted when different organs are selected in the GUI based on how the image quality scores of slices associated with the selected organs are aggregated, the image quality score may be adjusted without launching the image quality score generator, where the ML model is not used to generate a new image quality score. Rather, the image quality score may be adjusted while the image quality score generator is in an unlaunched state. By calculating the image quality scores in this manner, a responsiveness of the GUI to the user feedback may be increased, such that the user feedback may be quickly and easily provided and stored in the system.

In other words, the methods disclosed herein for generating, displaying, and adjusting the image quality scores and/or low-level image quality metrics improve the capabilities of the image quality assessment system by reducing an amount of processing that would otherwise be performed when regenerating image quality scores using the ML model. Image quality data is generated in a first step by the image quality score generator using the ML model, and the image quality data is aggregated and displayed in a second step during which the image quality score generator is not running. Thus, if the user wishes to view image quality scores of specific organs of the medical image, the image quality scores can be generated quickly with minimal processing. In this way, the GUI improves the way the image quality assessment system stores and retrieves data in memory to reduce resource consumption. A specific manner of displaying image quality information to the user based on a limited set of image data is described, such that the user is not burdened by time-consuming, iterative calculations, or by navigating through pages of image quality data amassed for different portions of the medical image. Because the user is not forced to scroll down or navigate through various layers of data to view image quality scores, a rapid and efficient process for collecting image quality feedback is enabled. As a result, ground truth data for refining the ML model may be collected in larger amounts and in shorter time frames than would be permitted by prior art systems and GUIs. Thus, the disclosed invention improves the efficiency of the image quality GUI specifically, and the image quality assessment system in general.

Further, the methods described herein may be more robust with respect to a use of contrast, in relation to alternative methods that rely on a Hounsfield segmentation, since the utilization of contrast during an exam may change Hounsfield values.

Further still, the image quality assessment system may be configured to optimize computational resources through strategic data sampling. Specifically, rather than processing all images in a given series for organ segmentation and quality assessment, the system selectively samples a portion of the images (e.g., 1 image every N images) while maintaining a uniform distribution across the series. For example, in a series containing 700 images, the system may sample 100 images for analysis, reducing computational overhead by approximately 2× while still providing statistically meaningful results. This sampling approach enables practical implementation of 3D segmentation models that might otherwise be computationally prohibitive. Additionally, the system supports multi-modality applications, allowing the same technical framework to be applied across different imaging modalities (e.g., MR) where appropriate AI segmentation models exist. The system's quality metrics may be normalized between 0 and 10 based on various scoping parameters of the clinical context, such as data from the same hospital, hospital group, or patient type, enabling meaningful comparisons within specific clinical environments. Furthermore, the system can link structure-specific noise scores with structure-specific radiation dose measurements, enabling more granular optimization of the image quality to radiation dose ratio compared to global series-level measurements. This technical architecture supports future integration with electronic health records (EHR) and Radiology Information Systems (RIS) to incorporate additional clinical context beyond protocol information, enabling even more precise targeting of quality assessment to specific clinical needs.

The technical effect of generating an image quality score for a medical image is that an accuracy of parameter settings for acquiring medical images in scan protocols may be increased. By increasing the accuracy of the parameter settings, a number of scans that may be performed on a patient may be reduced, since in an alternate scenario where the parameter settings are not ideal and the disclosed methods are not used, an operator of a scanner may perform repeated scans in a trial-and-error fashion to determine suitable parameter settings. The technical effect of displaying image quality scores and low-level image quality metrics for user-selected portions of a medical image is that image quality data sufficient for training or retraining an ML model to generate the image quality scores may be collected in a semi-automated manner, whereas currently the image quality data is collected and compiled manually.

In another representation, an image quality assessment system comprises a display device, the image quality assessment system being configured to display in a graphical user interface (GUI) of the display device a medical image, an image quality score indicating a quality of the medical image, the image quality score generated by an image quality score generator of the image quality assessment system; and a menu listing one or more low-level image quality metrics used to generate the image quality score; and additionally being configured to display in the GUI a low-level image quality metric summary that can be reached directly from the menu; wherein the low-level image quality metric summary displays one or more limited sets of data generated based on the low-level image quality metrics, each of the sets of data in the list being selectable to launch a visualization of the set of data; and wherein in response to a user of the image quality assessment system selecting a low-level image quality metric in the menu, the low-level image quality metric summary is displayed in the GUI while the image quality score generator is in an unlaunched state.

The disclosure also provides support for a method for an image quality assessment system, the method comprising: receiving medical image series data from a user of the image quality assessment system, generating an image quality score for a medical image included in the medical image series data, the image quality score generated using a trained machine learning (ML) model, displaying the medical image and the image quality score in a graphical user interface (GUI) on a display device of the image quality assessment system, receiving an adjusted image quality score of the medical image from the user via the GUI, and using the adjusted image quality score to retrain the ML model, wherein the image quality score is based on aggregated image quality scores calculated for one or more organs and/or anatomical structures in the medical image. In a first example of the method, the method further comprises: identifying the one or more organs and/or anatomical structures in the medical image based on a name of a protocol used to acquire the medical image series data, the name extracted from metadata included in a Digital Imaging and Communications in Medicine (DICOM) file of the medical image series data. In a second example of the method, optionally including the first example, identifying the one or more organs and/or anatomical structures in the medical image further comprises selecting a standard protocol code of a publicly available lexicon associated with the name of the protocol, and retrieving a list of names of the one or more organs and/or anatomical structures associated with the standard protocol code in the publicly available lexicon. In a third example of the method, optionally including one or both of the first and second examples, the publicly available lexicon is a RadLex lexicon. In a fourth example of the method, optionally including one or more or each of the first through third examples, the image quality score is an overall image quality score, wherein generating the overall image quality score comprises calculating image quality scores for the one or more organs and/or anatomical structures and aggregating the image quality scores for the one or more organs and/or anatomical structures to generate the overall image quality score, wherein calculating the image quality scores for the one or more organs and/or anatomical structures further comprises: segmenting the one or more organs and/or anatomical structures, to determine boundaries of the one or more organs and/or anatomical structures, for each organ of the one or more organs and/or anatomical structures: generating an organ mask, based on the boundaries of the organ, and applying the organ mask when calculating the image quality scores to calculate the image quality scores based on image data corresponding to the organ and not based on image data not corresponding to the organ. In a fifth example of the method, optionally including one or more or each of the first through fourth examples, segmenting the one or more organs and/or anatomical structures further comprises mapping the retrieved list of the names of the one or more organs and/or anatomical structures associated with the standard protocol code in the publicly available lexicon to a set of one or more corresponding names of organs and/or anatomical structures used by segmentation software of the image quality assessment system, and segmenting the one or more organs and/or anatomical structures based on the set of one or more corresponding names. In a sixth example of the method, optionally including one or more or each of the first through fifth examples, calculating the image quality scores for the one or more organs and/or anatomical structures further comprises: for each organ of the one or more organs and/or anatomical structures: associating one or more slices of the medical image with the organ and/or anatomical structure, calculating image quality scores for the organ in each slice associated with the organ, using a respective organ mask, and aggregating the image quality scores from each slice. In a seventh example of the method, optionally including one or more or each of the first through sixth examples, calculating image quality scores for the organ in each slice associated with the organ further comprises computing a plurality of low-level quality metrics for each slice associated with the organ, and aggregating the plurality of low-level quality metrics across the slices. In an eighth example of the method, optionally including one or more or each of the first through seventh examples, the plurality of low-level quality metrics include at least one of: a signal-to-noise ratio (SNR), a total amount of noise, a noise power spectrum (NPS), and a contrast-to-noise ratio (CNR). In a ninth example of the method, optionally including one or more or each of the first through eighth examples, the plurality of low-level quality metrics are inputs into the ML model. In a tenth example of the method, optionally including one or more or each of the first through ninth examples, the method further comprises: performing a first scan of a patient with a medical imaging system at a first imaging session, using a first set of acquisition parameters recommended by a second ML model based on data of the patient, to generate a first image volume of a first image quality, receiving an adjusted set of acquisition parameters from a user of the medical imaging system, and performing a second scan of the patient at the first imaging session, using the adjusted set of acquisition parameters to generate a second image volume of a second image quality, the second image quality higher than the first image quality, retraining the second ML model using training data including images ranked by users of the image quality assessment system as having a high image quality, and performing a third scan on the patient at a second imaging session after the first imaging session, using a third set of acquisition parameters recommended by the retrained second ML model based on the data of the patient, to generate a third image volume of a third image quality, wherein the third image quality is higher than the second image quality.

The disclosure also provides support for a medical imaging system comprising a display device, the medical imaging system being configured to display on the display device a menu listing a plurality of slices of a 3-D medical image viewable on the display device, and additionally being configured to display on the display device an image quality graphical user interface (GUI) that can be reached directly from the menu, wherein the image quality GUI displays, for a set of slices of the plurality of slices, an image quality score generated by an image quality score generator, and a limited list of results of low-level image quality metrics applied to the set of slices to generate the image quality score, the low-level image quality metrics applied to image data of one or more organs of each slice and not to image data outside the one or more organs using an organ mask, each result in the limited list being selectable to launch a display panel with additional information relating to the low-level image quality metrics applied to the set of slices and enable at least the selected result to be seen within the display panel, and wherein the image quality GUI is displayed while the image quality score generator is in an unlaunched state. In a first example of the system, the image quality score is based on aggregating the low-level image quality metrics over a plurality of organs and/or anatomical structures included in the plurality of slices of the 3-D medical image, and in response to a user selecting an organ or anatomical structure of the plurality of organs and/or anatomical structures via the image quality GUI, the image quality score displayed in the image quality GUI is updated while the image quality score generator is in the unlaunched state. In a second example of the system, optionally including the first example, the image quality score is indicated in the image quality GUI via an interactive graphical element including a needle, and the image quality score is adjustable by a user by adjusting a relative position of the needle within the interactive graphical element, and in response to the user adjusting the image quality score, the adjusted image quality score is stored in a memory of the medical imaging system. In a third example of the system, optionally including one or both of the first and second examples, the adjusted image quality score is used to retrain an ML model used by the image quality score generator to generate the image quality score.

The disclosure also provides support for an image quality assessment system, comprising: a processor, and a memory storing instructions that when executed, cause the processor to: receive a Digital Imaging and Communications in Medicine (DICOM) file including medical image series data acquired from a medical imaging system from a user of the image quality assessment system, extract a name of a protocol used to acquire the medical image series data from metadata of the DICOM file, perform a segmentation of a plurality of organs and/or anatomical structures included in the protocol to determine boundaries of the plurality of organs and/or anatomical structures, calculate an image quality score for each organ and/or anatomical structure of the plurality of organs and/or anatomical structures in each slice of each image of the medical image series data using a machine learning (ML) model, the ML model taking as input a plurality of low-level image quality metrics applied to each organ and/or anatomical structure of each slice, aggregate image quality scores of each organ and/or anatomical structure of the plurality of organs and/or anatomical structures to generate an overall image quality score of the medical image series data, display a medical image of the medical image series data to the user of the image quality assessment system via a graphical user interface (GUI) of the image quality assessment system, display the overall image quality score of the medical image in the GUI, receive an adjusted image quality score of the medical from the user via the GUI, and retrain the ML model using training data including the adjusted image quality score. In a first example of the system, further instructions are stored in the memory that when executed, cause the processor to: retrieve a standard protocol code associated with the name of the protocol from a radiology lexicon, retrieve a list of names of the plurality of organs and/or anatomical structures associated with the standard protocol code from the radiology lexicon, map the retrieved list of names to a second list of names of the plurality of organs and/or anatomical structures used by segmentation software of the image quality assessment system, and perform the segmentation of the plurality of organs and/or anatomical structures based on the second list of names. In a second example of the system, optionally including the first example, further instructions are stored in the memory that when executed, cause the processor to: for each organ and/or anatomical structure of the plurality of organs and/or anatomical structures: generate an organ mask based on the boundaries, and apply the organ mask when calculating the image quality score for the organ and/or anatomical structure, to calculate the image quality score based on image data corresponding to the organ and/or anatomical structure and not based on image data not corresponding to the organ and/or anatomical structure. In a third example of the system, optionally including one or both of the first and second examples, further instructions are stored in the memory that when executed, cause the processor to calculate the image quality scores for an organ of the organs and/or anatomical structures by computing a plurality of low-level quality metrics for each slice associated with the organ using the organ mask of the organ, and aggregating the plurality of low-level quality metrics across a plurality of slices. In a fourth example of the system, optionally including one or more or each of the first through third examples, further instructions are stored in the memory that when executed, cause the processor to: detect a selection of an organ in the medical image by the user, and in response, display an image quality score for the selected organ in the GUI.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc., are used merely as labels and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims and may include other examples that occur to those of ordinary skill in the art. Such other examples are intended to be within the scope of the claims if they have structural elements that do not differ from the literal language of the claims or if they include equivalent structural elements with insubstantial differences from the literal languages of the claims.

1. A method for an image quality assessment system, the method comprising:
receiving medical image series data from a user of the image quality assessment system;
generating an image quality score for a medical image included in the medical image series data, the image quality score generated using a trained machine learning (ML) model;
displaying the medical image and the image quality score in a graphical user interface (GUI) on a display device of the image quality assessment system;
receiving an adjusted image quality score of the medical image from the user via the GUI; and
using the adjusted image quality score to retrain the ML model;
wherein the image quality score is based on aggregated image quality scores calculated for one or more organs and/or anatomical structures in the medical image.

2. The method of claim 1, further comprising identifying the one or more organs and/or anatomical structures in the medical image based on a name of a protocol used to acquire the medical image series data, the name extracted from metadata included in a Digital Imaging and Communications in Medicine (DICOM) file of the medical image series data.

3. The method of claim 2, wherein identifying the one or more organs and/or anatomical structures in the medical image further comprises selecting a standard protocol code of a publicly available lexicon associated with the name of the protocol, and retrieving a list of names of the one or more organs and/or anatomical structures associated with the standard protocol code in the publicly available lexicon.

4. The method of claim 3, wherein the publicly available lexicon is a RadLex lexicon.

5. The method of claim 3, wherein the image quality score is an overall image quality score, wherein generating the overall image quality score comprises calculating image quality scores for the one or more organs and/or anatomical structures and aggregating the image quality scores for the one or more organs and/or anatomical structures to generate the overall image quality score, wherein calculating the image quality scores for the one or more organs and/or anatomical structures further comprises:
segmenting the one or more organs and/or anatomical structures, to determine boundaries of the one or more organs and/or anatomical structures;
for each organ of the one or more organs and/or anatomical structures:
generating an organ mask, based on the boundaries of the organ; and
applying the organ mask when calculating the image quality scores to calculate the image quality scores based on image data corresponding to the organ and not based on image data not corresponding to the organ.

6. The method of claim 5, wherein segmenting the one or more organs and/or anatomical structures further comprises mapping the retrieved list of the names of the one or more organs and/or anatomical structures associated with the standard protocol code in the publicly available lexicon to a set of one or more corresponding names of organs and/or anatomical structures used by segmentation software of the image quality assessment system, and segmenting the one or more organs and/or anatomical structures based on the set of one or more corresponding names.

7. The method of claim 5, wherein calculating the image quality scores for the one or more organs and/or anatomical structures further comprises:
for each organ of the one or more organs and/or anatomical structures:
associating one or more slices of the medical image with the organ and/or anatomical structure;
calculating image quality scores for the organ in each slice associated with the organ, using a respective organ mask; and
aggregating the image quality scores from each slice.

8. The method of claim 7, wherein calculating image quality scores for the organ in each slice associated with the organ further comprises computing a plurality of low-level quality metrics for each slice associated with the organ, and aggregating the plurality of low-level quality metrics across the slices.

9. The method of claim 8, wherein the plurality of low-level quality metrics includes at least one of:
a signal-to-noise ratio (SNR);
a total amount of noise;
a noise power spectrum (NPS); and
a contrast-to-noise ratio (CNR).

10. The method of claim 8, wherein the plurality of low-level quality metrics are inputs into the ML model.

11. The method of claim 1, further comprising:
performing a first scan of a patient with a medical imaging system at a first imaging session, using a first set of acquisition parameters recommended by a second ML model based on data of the patient, to generate a first image volume of a first image quality;
receiving an adjusted set of acquisition parameters from a user of the medical imaging system, and performing a second scan of the patient at the first imaging session, using the adjusted set of acquisition parameters to generate a second image volume of a second image quality, the second image quality higher than the first image quality;
retraining the second ML model using training data including images ranked by users of the image quality assessment system as having a high image quality; and
performing a third scan on the patient at a second imaging session after the first imaging session, using a third set of acquisition parameters recommended by the retrained second ML model based on the data of the patient, to generate a third image volume of a third image quality;
wherein the third image quality is higher than the second image quality.

12. A medical imaging system comprising a display device, the medical imaging system being configured to display on the display device a menu listing a plurality of slices of a 3-D medical image viewable on the display device; and additionally being configured to display on the display device an image quality graphical user interface (GUI) that can be reached directly from the menu; wherein the image quality GUI displays, for a set of slices of the plurality of slices, an image quality score generated by an image quality score generator, and a limited list of results of low-level image quality metrics applied to the set of slices to generate the image quality score, the low-level image quality metrics applied to image data of one or more organs of each slice and not to image data outside the one or more organs using an organ mask, each result in the limited list being selectable to launch a display panel with additional information relating to the low-level image quality metrics applied to the set of slices and enable at least the selected result to be seen within the display panel, and wherein the image quality GUI is displayed while the image quality score generator is in an unlaunched state.

13. The medical imaging system of claim 12, wherein the image quality score is based on aggregating the low-level image quality metrics over a plurality of organs and/or anatomical structures included in the plurality of slices of the 3-D medical image; and
in response to a user selecting an organ or anatomical structure of the plurality of organs and/or anatomical structures via the image quality GUI, the image quality score displayed in the image quality GUI is updated while the image quality score generator is in the unlaunched state.

14. The medical imaging system of claim 12, wherein the image quality score is indicated in the image quality GUI via an interactive graphical element including a needle, and the image quality score is adjustable by a user by adjusting a relative position of the needle within the interactive graphical element; and
in response to the user adjusting the image quality score, the adjusted image quality score is stored in a memory of the medical imaging system.

15. The medical imaging system of claim 14, wherein the adjusted image quality score is used to retrain an ML model used by the image quality score generator to generate the image quality score.

16. An image quality assessment system, comprising:
a processor; and
a memory storing instructions that when executed, cause the processor to:
receive a Digital Imaging and Communications in Medicine (DICOM) file including medical image series data acquired from a medical imaging system from a user of the image quality assessment system;
extract a name of a protocol used to acquire the medical image series data from metadata of the DICOM file;
perform a segmentation of a plurality of organs and/or anatomical structures included in the protocol to determine boundaries of the plurality of organs and/or anatomical structures;
calculate an image quality score for each organ and/or anatomical structure of the plurality of organs and/or anatomical structures in each slice of each image of the medical image series data using a machine learning (ML) model, the ML model taking as input a plurality of low-level image quality metrics applied to each organ and/or anatomical structure of each slice;
aggregate image quality scores of each organ and/or anatomical structure of the plurality of organs and/or anatomical structures to generate an overall image quality score of the medical image series data;
display a medical image of the medical image series data to the user of the image quality assessment system via a graphical user interface (GUI) of the image quality assessment system;
display the overall image quality score of the medical image in the GUI;
receive an adjusted image quality score of the medical from the user via the GUI; and
retrain the ML model using training data including the adjusted image quality score.

17. The image quality assessment system of claim 16, wherein further instructions are stored in the memory that when executed, cause the processor to:
retrieve a standard protocol code associated with the name of the protocol from a radiology lexicon;
retrieve a list of names of the plurality of organs and/or anatomical structures associated with the standard protocol code from the radiology lexicon;
map the retrieved list of names to a second list of names of the plurality of organs and/or anatomical structures used by segmentation software of the image quality assessment system; and
perform the segmentation of the plurality of organs and/or anatomical structures based on the second list of names.

18. The image quality assessment system of claim 16, wherein further instructions are stored in the memory that when executed, cause the processor to:
for each organ and/or anatomical structure of the plurality of organs and/or anatomical structures:
generate an organ mask based on the boundaries; and
apply the organ mask when calculating the image quality score for the organ and/or anatomical structure, to calculate the image quality score based on image data corresponding to the organ and/or anatomical structure and not based on image data not corresponding to the organ and/or anatomical structure.

19. The image quality assessment system of claim 18, wherein further instructions are stored in the memory that when executed, cause the processor to calculate the image quality scores for an organ of the organs and/or anatomical structures by computing a plurality of low-level quality metrics for each slice associated with the organ using the organ mask of the organ, and aggregating the plurality of low-level quality metrics across a plurality of slices.

20. The image quality assessment system of claim 18, wherein further instructions are stored in the memory that when executed, cause the processor to:
detect a selection of an organ in the medical image by the user, and in response, display an image quality score for the selected organ in the GUI.
